(19) United States
(12) Patent Application Publication
(10) Pub. No.: US 2005/0101674 A1
(43) Pub. Date: May 12, 2005

(54) PPMP as a ceramide catabolism inhibitor for cancer treatment

(76) Inventors: Barry James Maurer, Sylmar, CA (US); Charles Patrick Reynolds, Sherman Oaks, CA (US)

Correspondence Address: PERKINS COIE LLP, POST OFFICE BOX 1208, SEATTLE, WA, 98111-1208, US

(21) Appl. No.: 10/712,763

(22) Filed: Nov. 12, 2003

(51) Int. Cl. A61K 31/16; A61K 31/225

(52) U.S. Cl. 514/625

(57) ABSTRACT

The present invention relates to a method of treating a hyperproliferative disorder comprising administering a ceramide generating retinoid comprising a retinoic acid derivative or a pharmaceutically acceptable salt thereof, and D-threo-PPMP as a ceramide degradation inhibitor or a pharmaceutically acceptable salt thereof, wherein the hyperproliferative disorder is a tumor; and wherein the ceramide generating retinoid is administered in an amount effective to produce necrosis, apoptosis or both in the tumor, and the ceramide degradation inhibitor is administered in an amount effective to increase the necrosis, apoptosis or both in the tumor over that expected to be produced by the sum of that produced by the ceramide generating retinoid and the ceramide degradation inhibitor when administered separately.

### BACKGROUND OF THE INVENTION

### FIELD OF THE INVENTION

The invention generally relates to the treatment of hyperproliferative disorders such as tumors.

### BACKGROUND

It is estimated that there are approximately 1,300,000 new cases of cancer in children and adults in the United States annually, resulting in over 550,000 deaths. These cancers include cancers of the genital system, the digestive system, the respiratory system, the breast, the urinary system, the skin, the oral cavity and pharynx, the endocrine system, the brain and nervous system, of soft issues, of the bones and joints, of the eye and orbit, of the lymph glands (such as lymphomas), and of the blood (such as leukemias). Thus, cancer is the second most common cause of death in the United States.

Accordingly there is a need for improved therapies for the treatment of such cancers.

### SUMMARY OF THE INVENTION

It is an object of the present invention to provide agents and methods for use of said agents for improved efficacy of chemotherapy for multiple cancers.

This and other aspects of the present invention which may become obvious to those skilled in the art through the following description of the invention are achieved by a method of treating a hyperproliferative disorder comprising administering a ceramide-generating anticancer agent or treatment, and a ceramide degradation inhibitor or a pharmaceutically acceptable salt thereof, wherein the hyperproliferative disorder is a tumor, and wherein the ceramide-generating anticancer agent or treatment is administered in an amount effective to produce necrosis, apoptosis or both in the tumor, and the ceramide degradation inhibitor is administered in an amount effective to increase the necrosis, apoptosis or both in the tumor over that expected to be produced by the sum of that produced by the ceramide-generating anticancer agent or treatment and the ceramide degradation inhibitor when administered separately.

This and other aspects of the present invention are also achieved by formulations for treating a hyperproliferative disorder comprising a ceramide-generating anticancer agent or treatment, and a ceramide degradation inhibitor or a pharmaceutically acceptable salt thereof, wherein the hyperproliferative disorder is a tumor, and wherein the ceramide generating retinoid is administered in an amount effective to produce necrosis, apoptosis or both in the tumor, and the ceramide degradation inhibitor is administered in an amount effective to increase the necrosis, apoptosis or both in the tumor over that expected to be produced by the sum of that produce by the ceramide-generating anticancer agent or treatment and the ceramide degradation inhibitor when administered separately.

### BRIEF DESCRIPTION OF THE DRAWINGS

FIG. 1 shows partial metabolic pathways of ceramide.

FIG. 2 shows 4-HPR cytotoxicity correlated with ceramide increase.

FIG. 3 shows that 4-HPR-induce ceramide is cytotoxic, specifically L-cyvloserine, an inhibitor of de novo ceramide synthesis, decreases the cytotoxicity of 4-HPR and 4-HPR with safingol.

FIG. 4 shows that 4-HPR-induced ceramide is cytotoxic, specifically, overexpression of Glucosylceramide synthase (GSC) decreased 4-HPR cytotoxi city and abrogated the cytotoxic synergy of safingol in MCF-7 breast cancer cells.

FIG. 5 shows that GSC and 1-O-ACS are expressed in neurobiastoma and leukemia cell lines and are therefore targets for therapeutic intervention.

FIG. 6 shows that D,L-threo-PPMP synergizes 4-HPR cytotoxicity in a resistant neuroblastoma cell line.

FIG. 7 shows that D,L-threo-PPMP increases 4-HPR induced ceramide in a multi-drug-resistant-neuroblastoma cell line.

FIG. 8 shows that D,L-threo-PPMP synergized 4-HPR cytotoxicity in ALL cell lines.

FIG. 9 shows that D-threo-PPMP increases ceramide more than L-threo-PPMP.

FIG. 10 shows that D-threo-PPMP more potently synergizes 4-HPR cytotoxicity in a neuroblastoma cell line.

FIG. 11 shows that D-threo-PPMP more potently synergizes 4-HPR cytotoxicity in a prostate cell line.

FIG. 12 shows a possible method of synthesis of D-threo-PPMP.

FIG. 13 shows the continuous venous infusion of 4-HPR in rats.

### DETAILED DESCRIPTION OF THE INVENTION

In order to fully understand the manner in which the above-recited details and other advantages and objects according to the invention are obtained, a more detailed description of the invention will be rendered by reference to specific embodiments thereof.

Many cancer chemotherapeutic drugs in current clinical use directly or indirectly damage DNA, leading to cell death mostly via p53-dependent apoptosis. Tumor cells that do not have functional p53 (approximately ½ of adult cancers, and many relapsed childhood cancers) show, at best, modest responses to p53-dependent chemotherapeutic agents. Even in those childhood cancers that are highly responsive to chemotherapy, where a cure can lead to many years of extended life span, the mutagenic potential of current chemotherapy creates a high risk of secondary malignancies. Thus, developing a chemotherapy that is cytotoxic for malignant cells without causing DNA damage and that is p53-independent, offers the potential to by-pass common mechanisms of drug-resistance and to diminish both early and late side-effects. One approach that fits this description is the selective overproduction of the pro-death lipid, ceramide, in cancer cells. Fenretinide is as an agent that stimulates ceramide production in malignant cells, but not in normal cells. Doxorubicin is another example of a chemotherapeutic agent that can increase ceramide in cancer cells. An important component of such a strategy is to develop drugs that diminish the ability of tumor cells to detoxify ceramide, and we here demonstrate that one such drug is D-threo-stereoisomer of 1-phenyl-2-palmitoylamino-3-morpholino-1-propanol (PPMP).

PPMP is an inhibitor of ceramide catabolism, and as such, can enhance the anti-cancer activity of the cytotoxic retinoid, fenretinide (4-HPR). We have found: 1) that fenretinide, significantly increases ceramide via de novo synthesis in solid tumor and acute leukemia cell lines of both pediatric and adult cancers in a dose- and time-dependent manner in vitro; and 2) that inhibitors of ceramide catabolism, such as PPMP, synergistically increases 4-HPR cytotoxicity, even in cell lines with alkylator resistance and/or lacking functional p53. Our studies indicate that especially D-threo-PPMP, an inhibitor of both glucosylceramide synthase and 1-O-acylceramide synthase, prevents the catabolism of 4-HPR-induced ceramide, which results in a synergistic increase of 4-HPR cytotoxicity in vitro. The stimulation and manipulation of de novo ceramide in vivo represents a totally novel form of chemotherapy. Accordingly, D-threo-PPMP will most likely enhance the anti-cancer effect of 4-HPR, and other ceramide-generating anticancer agents or treatments, in both pediatric and adult cancer patients with tolerable systemic toxicity. A ceramide-generating anticancer agent or treatment is any agent or treatment that directly or indirectly results in the increase in or generation of ceramide.

A method according to the present invention includes the use of a potentiating agent, such as D-threo-PPMP or pharmaceutically acceptable salts or esters thereof, as an inhibitor of ceramide catabolism in order to enhance the anti-cancer activity of the compound such as the cytotoxic retinoid fenretinide (4-HPR) by inhibiting or preventing the growth of tumors, cancers, neoplastic tissue and other premalignant and normeoplastic hyperproliferative or hyperplastic disorders. The method may be used to inhibit growth and/or induce cytotoxicity by necrotic or apoptotic mechanisms, or both, in the target cells which are generally hyperproliferative cells including tumors, cancers and neoplastic tissue along with premalignant and non-neoplastic or non-malignant hyperproliferative disorders.

Examples of tumors, cancers and neoplastic tissue that can be treated by the present method include but are not limited to malignant disorders such as breast cancers, osteosarcomas, angiosarcomas, fibrosarcomas and other sarcomas, leukemias, lymphomas, sinus tumors, ovarian, uretal, bladder, prostate and other genitourinary cancers, colon, esophageal and stomach cancers and other gastrointestinal cancers, lung cancer, myelomas, pancreatic cancers, liver cancers, kidney cancers, endocrine cancers, skin cancers, and brain or central and peripheral nervous system tumors, malignant or benign, including gliomas and neuroblastomas.

Examples of pre-malignant and non-malignant hyperproliferative disorders include but are not limited to myelodysplastic disorders, cervical carcinoma-in-situ, familial intestinal polyposes such as Gardner's syndrome, oral leukoplakias, histiocytosis, keloids, hemangiomas, hyperproliferative arterial stenosis, inflammatory arthritis, hyperkeratosis and papulosquamous eruptions including arthritis. Also included are viral induced hyperproliferative diseases such as warts and EBV induced disease such as infectious mononucleosis, scar formation and the like. The method may be employed with any subject known or suspected of carrying or at risk of developing a hyperproliferative disorder.

Treatment of a hyperproliferative disorder refers to methods of killing inhibiting or slowing the growth or increase in size of a body or population of hyperproliferative cell numbers or preventing spread to other anatomical sites as well as reducing the size of a hyperproliferative growth or numbers of hyperproliferative cells. Treatment is not necessarily meant to imply a cure or complete abolition of hyperproliferative growths. A treatment effective amount is an amount effective to result in the killing, the slowing of the rate of growth of hyperproliferative cells the decrease in the size of a body of hyperproliferative cells, and or the reduction in number of hyperproliferative cells. The potentiating agent or agents are included in an amount sufficient to enhance the activity of the first compound such that the two or more compounds together have a greater therapeutic efficacy than the individual compounds administered alone.

The administration of the two or more compounds in combination means that the two compounds are administered closely enough in time that the presence of one alters the biological effects of the other. The two compounds may be administered simultaneously or sequentially. Simultaneous administration may be carried out by mixing the compounds prior to administration or by administering the compounds at the same point in time but at different anatomical sites or using different routes of administration.

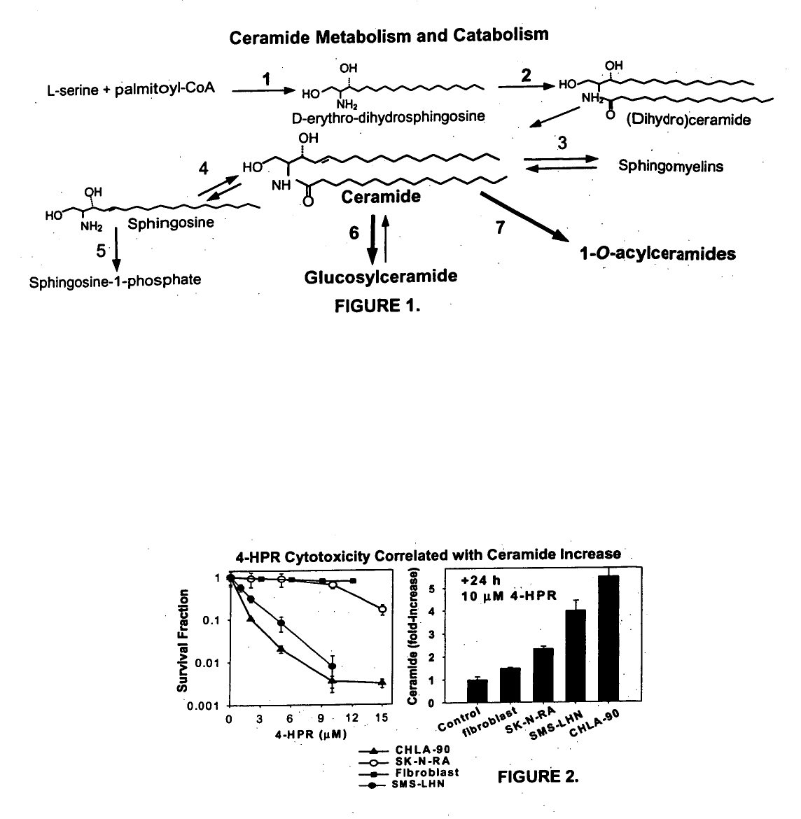

Administration of the compounds affect ceramide levels in a patient. Ceramide is a sphingolipid precursor of sphingomyelin and glucosphingolipids. Referring to FIG. 1, ceramide is generated in different cellular compartments by de novo synthesis, or from sphingomyelin breakdown under the action of sphingomyelinases. Ceramide levels are tightly controlled by regulation of de novo synthesis and/or the shunting of ceramide into nontoxic lipid fractions, such as glucosylceramide, 1-O-acylceramide, and sphingomyelin. Ceramide is also metabolized to sphingosine by various ceramidases. Ceramide has been implicated as a second messenger in several death-signaling pathways, including TNF-alpha, Fas, radiation treatment, certain chemotherapeutic agents, and thermal shock. Cellular responses to ceramide depend upon its cellular compartment. While the role of sphingomyelin-derived ceramide in death signaling is being clarified, current data support a cytotoxic function of ceramide derived from de novo synthesis. Ceramide has been reported to disrupt electron transport in mitochondria, leading to the generation of reactive oxygen species (ROS), and ceramide can be generated as a consequence of apoptosis (caspase) activation. Ceramide has been reported to initiate cell death under hypoxic conditions in a p53-independent manner. Ceramide has been shown to activate multiple kinases whose activities impact upon cell death signaling/responses, including activation of caspases, ERKl/2 (pro-life), and JNK/SAPK (pro-death), phosphatases, and may inactivate cyclin-dependent kinase Cdk2 and telomerase activity.

Referring to FIG. 1, partial metabolic pathways of ceramide are described wherein de novo ceramide synthesis, Serine palmityoltransferase (SPT), inhibited by L-cycloserine, catalyzes the condensation of serine and palmitoyl-CoA to keto-sphinganine, which is reduced to D-erythro-dihydrosphingosine (sphinganine) 1. Sphinganine is acylated by (dihydro)ceramide synthase (CS), inhibited by fumonisin B, to dihydroceramide, which is then desaturated to ceramide 2. Alternatively, sphingomyelin is hydrolyzed by various sphingomyelinases to ceramide 3. Ceramide is catabolized to sphingosine by various ceramidases 4. Sphingosine is phosphorylated by sphingosine kinase (SK) to sphingosine-1-phosphate (S-1-P) 5. Ceramide is catabolized to glucosylceramides by Golgi-derived glucosylceramide synthase (inhibited by D,L-threo-PPMP) 6, or to 1-O-acylceramides by 1-O-acylceramide synthase (human lecithin: cholesterol acyltransferase-like lysophospholipase) (inhibited by D,L-erythro-, and D,L-threo-PPMP) 7.

Many studies of ceramide-mediated cytotoxicity have employed exogenous, short-chain, cell-penetrating ceramides, such as C2- or C6-ceramide, which may artificially violate ceramide compartmentalization. Importantly, it has been demonstrated that the sphingosine backbone of C2 and C6-ceramides, and therefore possibly of all short chain ceramides, are recycled into endogenous long chain ceramides and other sphingolipids through the action of a ceramidase and (probably golgi) ceramide synthase. This finding complicates the interpretation of data derived using short chain ceramides, but supports the cytotoxic role of de novo synthesized ceramide.

Inhibitors of ceramide catabolism are now described. Ceramide is degraded by various ceramidases, and catabolized to sphingomyelin, glucosylceramide, and 1-O-acylceramides as shown in FIG. 1. The cytotoxicity of ceramide can be increased by inhibitors that decrease its degradation and catabolism. Such inhibitors include certain stereoisomers of lphenyl-2-palmitoylamino-3-morpholino-1-propanol (PDMP) and its more active homolog PPMP, which inhibit of glucosylceramide synthase and 1-O-acylceramide synthase. The cytotoxicity of the ceramide-increasing retinoid, fenretinide (4-HPR), is synergized by these agents in multiple solid tumor and leukemia cell lines in vitro. Inhibition of glucosylceramide formation may also been found to reverse drug resistance to doxorubicin in the MCF7 breast cancer cell line. Indeed, inhibition of glucosylceramide synthesis (GCS), or poly-drug elevation of ceramide, may be a potential chemotherapy. Unfortunately, clinically-available agents reported to inhibit GCS activity, such as tamoxifen, cyclosporine, and verapamil, do so in vitro only at levels that are not achievable in children or in most adult patients. More importantly, there are no known anticancer agents in clinical use that inhibit GCS and 1-O-acylceramide simultaneously. Thus, as inhibition of ceramide catabolism could enhance the efficacy of cancer chemotherapeutic agents acting via ceramide (such as 4-HPR), there is a need to develop new agents capable of inhibiting ceramide catabolism for clinical use.

PPMP is a homolog of the GCS and 1-O-ACS inhibitor, 1-phenyl-2-[decanoylamino]-3; morpholino-1-propanol (PDMP). PPMP, like its progenitor compound PDMP, has two chiral carbons and therefore, four stereoisomers: D-threo-PPMP; L-threo-PPMP; D-erythro-PPMP; and L-erythro-PPMP. PPMP is more commonly used in its racemic mixture forms of D,L-threo-PPMP and D,L-erythro-PPMP and these racemic mixtures are commonly called “PPMP”. PPMP is 10 to 20 times more active in intact cells than is PDMP. PDMP was originally developed as an inhibitor of glucosylceramide synthase (GCS) for the treatment of glycosphingolipid storage disorders, such as Gaucher's disease. Unlike other glucosylceramide synthase inhibitors, however, such as N-butyldeoxynojirimycin (NB-DNJ), PPPP (P4), or 4′-Hydroxy-P4, D-threo-PDMP has been shown to increase endogenous ceramide in association with a dose-dependent reduction of growth of treated cells. As such, D-threo-PDMP is a less desirable agent for the treatment of storage disorders, but this growth-inhibitory property has been used as a well-tolerated chemotherapy to treat Ehrlich ascites tumor cells in mice, C6 glioma cells in rat models, and decrease murine Lewis lung carcinoma metastasis.

We demonstrate that D-threo-PPMP is the superior stereoisomer of PPMP for inhibiting the degradation of ceramide and increasing the anticancer activity of ceramide-increasing anticancer agents, such as fenretinide, thus making it preferred to any other PPMP compound for this purpose. D-threo-PDMP may derive its ability to increase ceramide from its ability to simultaneously inhibit 1-O-acylceramide synthase (lecithin:cholesterol acyltransferase-like lysophospholipase) and glucosylceramide synthases (GCS). 1-O-acylceramide synthase (1-O-ACS) is a recently characterized enzyme capable of acylating ceramides at the carbon-1 position and is widely expressed. Transacylation is postulated to act as a metabolic buffer for cells under ceramide stress, allowing ceramide to be stored nontoxically for future metabolism. D,L-erythro-PDMP does not inhibit GCS, but also causes ceramide accumulation and growth inhibition, and thus likely inhibits 1-O-ACS. We also demonstrate that, unexpectedly, L-threo-PPMP does not inhibit the formation of glucosylceramide and therefore that the racemic mixture D,L-PPMP has less activity than D-threo-PPMP in preventing ceramide catabolism and increasing ceramide levels. Interestingly, L-threo-PDMP does not inhibit GCS, but rather stimulates glycosphingolipid biosynthesis.

Additionally, PDMP and PPMP reverse the P-glycoprotein-mediated multidrug resistance (MDR) phenotype in MCF-7 breast cancer cells, KGla and K562 leukemia cells, and KB cervical carcinoma cells. They also enhance doxorubicin-induced apoptosis in MCF-7 breast cancer and HepG2 hepatoma cells, and synergize taxol and vincristine cytotoxicity in neuroblastoma cells in association with increased ceramide.

We have found that D-threo-PPMP is effective as a GCS and 1-O-ACS inhibitor for use in combination with the cytotoxic, ceramide-increasing retinoid, fenretinide (4-HPR), recognizing that it may also synergize other chemotherapeutic agents and treatments as described above.

Fenretinide

The synthetic retinoid (vitamin A-derivative), N-(4-hydroxyphenyl)retinamide (fenretinide, 4-HPR), has been shown to be cytotoxic to a variety of cancer cell lines in vitro, including neuroblastoma, colorectal, head and neck, breast, prostate, lung, ovarian, cervical, pancreas, and leukemia/lymphoma, at 4-HPR concentrations of 1-12 μM. 4-HPR induces cell death by apoptosis, necrosis, or mixed apoptosis/necrosis. 4-HPR has been reported to be cytotoxic in a p53-independent manner in cell lines of leukemia/lymphoma, and in small cell and non-small cell lung cancer. 4-HPR may also induce cell death in a p53- and caspase-independent manner by mixed apoptosis/necrosis in neuroblastoma cell lines. Cell death was delayed, but still occurred, in leukemia cells that over-expressed Bcl-2. Induction of apoptosis by 4-HPR in prostate and breast cancer cell lines coincides with induction of TGF-β. 4-HPR cytotoxicity is associated with c-Jun N-terminal kinase (JNK) activation in PC-3 prostate carcinoma cells.

Clinically, low-dose oral 4-HPR (200-900 mg/day; 1 to 3 μM plasma levels) has been studied as a chemopreventative agent in breast, bladder, cervical, bronchial, melanoma, and oral cavity cancers, with minimal toxicity, but with minimal reported success. A 30% reduction, however, in premalignant oral lesions (leukoplakia), and a reduction in contralateral breast cancer and ovarian cancer, have been reported using low-dose 4-HPR.

Phase I clinical trials of high-dose oral 4-HPR in adult and pediatric solid tumors have produced the following results. In pediatrics, the maximally tolerated dose (MTD) of oral 4-HPR administered for 7 days, every 3 weeks, was 2475 mg/m²/day, which achieved 4-HPR plasma levels of 6 to 10 μM with minimal systemic toxicity. Similar results, but with lower plasma levels, were observed in the adult high-dose oral 4-HPR study, with a recommended ‘practical’ dose for Phase II studies of 1800 mg/m²/day. Poor absorption of the currently available oral 4-HPR formulation appear to be a major dosing limitation in both studies.

The mechanism of 4-HPR cytotoxicity is complex. 4-HPR has significant retinoid receptor-independent cytotoxicity. Reactive oxygen species (ROS) contributed to 4-HPR cytotoxicity in HL-60 myeloid leukemia cell lines, in cervical and squamous cell carcinoma cells, and 4-HPR increases ROS in neuroblastoma cell lines. ROS was detected in five head and neck, and five lung cancer cell lines, but antioxidants only blocked 4-HPR-induced apoptosis in two of these cell lines. Thus, ROS is linked to 4-HPR exposure but its exact contribution to cytotoxicity in all cases is not clear.

Further, 4-HPR may cause large, novel increases of ceramide in cell lines of susceptible neuroblastoma, leukemia, and PNET/Ewing's sarcoma, in vitro, in a time- and dose-dependent manner, by the stimulation of de novo synthesis. Significantly, 4-HPR is nontoxic, and minimally increased ceramide, in normal fibroblasts and peripheral blood mononuclear cells, and was nontoxic in marrow myeloid progenitors. There is a striking synergism of 4-HPR cytotoxicity by modulators of ceramide catabolism or activity, such as D,L-threo-PPMP and safingol, in cancer cell lines of neuroblastoma, lung, melanoma, prostate, colon, breast, and the pancreas, including those with p53 mutations and/or high level alkylator-resistance. D,L-threo-PPMP, an inhibitor of glucosylceramide and 1-O-acylceramide synthases, further increases 4-HPR-induced ceramide levels and cytotoxicity in 4 of 6 acute lymphoblastic leukemia (ALL) cell lines. In the following figures, D,L-threo-PPMP is shown to further increase ceramide levels and cytotoxicity in 4-HPR-exposed prostate cancer cell lines. Notably, the cytotoxicity observed in 4-HPR-containing drug combinations was at dose levels which were nontoxic to normal fibroblasts and bone marrow myeloid progenitors. Interestingly, 4-HPR is nontoxic, and minimally increased ceramide, in an immortalized (but not transformed) rapidly proliferating B cell lymphoblastoid cell line, supporting the apparent malignancy-specific nature of 4-HPR cytotoxicity and ceramide induction.

A 4-HPR-based therapy, with its novel ceramide-based mechanism of action, may be effective against many solid tumors and hematopoetic malignancies (such as leukemias and lymphomas) that are resistant to existing therapies, and may be easily incorporated into current treatment regimens. Many cancer chemotherapy treatments are limited in efficacy by undesirable side effects in the body, especially toxicity to normal blood-forming cells in the bone marrow (i.e. myelotoxicity). Myelotoxicity can limit how much of the anticancer drug(s) that can be delivered for an anticancer effect, and necessitate blood transfusions of red blood cells and platelets, and predispose the patient to infections. A chemotherapy that is minimally myelotoxic therefore has distinct advantages. For example, if Phase I trials confirm that high-dose 4-HPR, and 4-HPR+D-threo-PPMP are minimally myelotoxic, then they could be considered for a Phase II window in the Consolidation, or Interim Maintenance, phases of current high risk acute lymphoblastic leukemia (ALL) protocols, and for inclusion post-myeloablative therapy in neuroblastoma. This would mount a ceramide-based attack in a setting of minimal residual disease, hopefully prior to the expansion of resistant disease clones. Additionally, courses of minimally myelotoxic 4-HPR-based therapies could be employed late in the prolonged marrow recovery phases of current acute myelogenous leukemia (AML) therapies. Alternatively, should 4-HPR-based therapies have moderate myelotoxic effects, making it less desirable to incorporate them into early treatment phases, they could be employed during or after ALL maintenance phases, or after marrow recovery from the last courses of current AML therapies, or pre-myeloablative therapy in neuroblastoma. Additionally, such minimally-toxic ceramide-based chemotherapies could be combined before or after current therapies for many solid tumors.

Further, 4-HPR and PPMP may have anticancer activity in at least several adult malignancies, including colon, breast, and prostate cancer.

The treatment of hyperproliferative disorders with a retinoid and a ceramide degradation inhibitor is generally described in U.S. Pat. Nos. 6,352,844 and 6,368,831 to Maurer et al. which are incorporated herein by reference.

Further, as set forth in the following examples, D-threo-PPMP has unexpectedly been found to be most effective in inhibit the catabolism of 4-HPR-induced ceramide and synergize 4-HPR cytotoxicity when compared to L-threo-PPMP and D,L-erythro-PPMP and when used in the present method. Further, as PPMP is 10-20 times more active than PDMP, D-threo-PPMP is unexpectedly the most preferred stereoisomer of all PPMP and PDMP compounds.

In addition, the use of D-threo-PPMP satisfies the need for a single drug agent that is pure (i.e., not a racemic mixture), where the activity of the drug agent can be ascribed to a single molecular entity, rather than unknown contributions from two molecular entities, thus greatly simplifying pharmacokinetic and pharmacodynamic effects which may affect anticancer efficacy, as well as, greatly simplify regulatory considerations for the U.S. Food and Drug Administration. Further D-threo-PPMP exhibits simultaneous inhibitory activities against both GCS and ACS and as a result will effect improved efficacy of chemotherapy for multiple cancers.

Synthesis of D-threo-PPMP

Referring to FIG. 12, the synthesis of D-threo-PPMP may be via stereoselective addition of phenyl cuprate to D-Gamer aldehyde. The syn adduct, which leads to the D-threo isomer, will be the major product. The minor L-erythro isomer (approximately 5%) can be removed by crystallization from chloroform at the late stage of the synthesis. Synthesis starts with a four-step synthetic procedure for the production of D-Garner Aldehyde from D-serine. We have synthesized L-Gamer Aldehyde from L-serine at the kilogram scale utilizing the same method for the production of safingol. Garner Aldehyde was obtained in 28% overall yield at 98+% ee purity without chromatography. Phenyl cuprate will be generated in situ by the reaction of copper(I) iodide and phenyl magnesium bromide. The addition of phenyl cuprate to D-Garner Aldehyde will yield intermediate 6. The deprotection of intermediate 6 with HCl produces D-threo-1-phenyl-2-amino-propane-1,3-diol-7. The intermediate 7 is reacted with activated palmitic acid and followed by base hydrolysis to form intermediate 8. The primary hydroxy group of intermediate 8 will be converted to the mesylate, and then substituted with morpholine to yield the final product D-threo-PPMP. Our synthetic plan is an efficient, practical synthesis to the enantiomerically pure PPMP. Most of the reactions described have been successfully conducted at the kilogram scale in our kilo lab. It is anticipated that the initial 2 g bach can be delivered within 6 to 8 weeks after the desired PPMP enantiomer is identified. Modifications to the current method of synthesis and other methods of synthesis of D-threo-PPMP are readily known to one of skill in the art.

Formulation and Administration

The active compounds may be formulated for administration in a single pharmaceutical carrier or in separate pharmaceutical carriers for the treatment of a variety of conditions. The carrier must be compatible with any other ingredients in the formulation and must not be deleterious to the patient. The carrier may be a solid or liquid or both and is preferably formulated with the compound as a unit dose formulation, such as a tablet which may contain 0.5% to 95% by weight of the active compound. One or more active compounds may be incorporated into the formulation which may be prepared by any of the known techniques of pharmacy consisting essentially of admixing the components and optionally including one or more accessory ingredients.

The formulations of the present invention are those suitable for oral, rectal, buccal (e.g., sub-ligual), vaginal, parenteral (e.g., subcutasneous, intramuscular, intradermal, or intravenous), topical (both skin and mucosal surfaces, including airway surfaces) and transdermal administration, although the most suitable route in any given case will depend on the nature and severity of the condition being treated and on the nature of the particular active compound being used.

Formulations suitable for oral administration may be presented in discrete units such as capsules cachets, lozenges, or tablets each containing a predetermined amount of the active compound(s), as a powder or granules, as a solution or a suspension in an aqueous or non-aqueous liquid, or as an oil-in-water or water-in-oil emulsion or a liposomal formulation. Such formulations may be prepared by any suitable method of pharmacy which includes the step of bringing into association the active compound and a suitable carrier (which may contain one or more accessory ingredients). In general, formulations are prepared by uniformly and intimately admixing the active compound with a liquid or finely divided solid carrier, or both, and then if necessary shaping the resulting mixture. For example a tablet may be prepared by compressing or molding a powder or granules containing the active compound(s), optionally with one or more accessory ingredients. Other delivery formulations may suggest themselves to one skilled in the art.

The therapeutically effective dosages of any one active ingredient will vary somewhat from compound to compound, patient to patient, and will depend upon factors such as the condition of the patient and the route of delivery. Such dosages can be determined in accordance with known pharmacological procedures in light of the disclosure herein.

For fenretinide for systemic treatment, a dose to achieve a plasma level of about 1, 2, or 3 μM to 10 or 20 μM, or 100 μM, will be employed; typically (for oral dosing) 50, 100, 500, 1000, 2000, or 3000 mg/m2 body surface area per day.

PDMP, the parental drug of PPMP, has been tested extensively in animals, is well-tolerated, and is capable of depleting glucosylceramide in vivo. The half-life of PDMP is approximately 1 hour and it is metabolized by the P-450 system. Despite it's superior activity (10 to 20 times as active), similar studies have not been reported for PPMP. PDMP is reported to fall out of aqueous solution in the absence of a nonionic detergent, like Myrj 52, but this detergent deposits in the liver in rodents. We have successfully solubilized PPMP in Diluent-12 for intravenous and intraperitoneal delivery to mice. Further, LYM-X-SORB technology (as described in U.S. Pat. No. 4,874,795, incorporated herein by reference), a non-liposomal, lipid-based, oral drug delivery system capable of solubilizing relatively insoluble drugs, including 4-HPR, has the potential to formulate PPMP for oral delivery. The LYM-X-SORB vector has proven well-tolerated in chronic administration in children with cystic fibrosis.

We have determined that high-dose 4-HPR+D-threo-PPMP given i.p. in mice is well-tolerated. We dissolved both 4-HPR and D-threo-PPMP to 15 mg/ml in NCI Diluent-12 (50/50 Cremophor EL/ethanol), which was diluted 1:3 in NS for injection. We co-injected 4-HPR at 125 mg/kg/day, with up to 125 mg/kg/day of D-threo-PPMP, in divided doses, i.p., for two courses of 5 days each, separated by a 10 day rest, with no obvious ill effects to the animals. Weights were stable. Mice survived >60+ days afterward. These results demonstrate that 4-HPR with D-threo-PPMP is well-tolerated in vivo.

Formulation of PPMP for Oral Delivery.

PPMP can be formulated for oral delivery using LYM-X-SORB™ technology (LYM-DRUG Products, LLC, a joint venture of AVANTI and BioMolecular Products, Inc.). The LYM-X-SORB (LXS) matrix is an oral drug delivery vehicle composed of FDA GRAS (generally regarded as safe) lipids: lysophosphatidylcholine (LPC), monoglyceride (MG), and free fatty acid (FA). The LXS monomeric matrix improves solubility and intestinal absorption of drugs by enfolding the drug into a LXS/drug complex at a 1:1 molar ratio. LPC:MG:FA ratios varying between 1:4:2 to 1:2:4 depending on the drug to be solubilized. The matrix can be liquid or solid at room temperature by varying the unsaturation of the fatty acids. LXS can solubilize poorly soluble compounds, such as retinoic acid, estradiol, cyclosporin A, diltiazem, and progesterone, among others. LXS is stable in physiological concentrations of sodium bicarbonate and sodium taurocholate (bile salt) and forms small particles in intestinal solutions (70 nm to less than 10 nm). The LXS matrix has proven safe in a one-year trial in children with cystic fibrosis. LXS may be used to formulate PPMP for oral delivery.

Several methods can be used for the incorporation of drugs into the pre-formed LXS eutectic matrix. The molar composition of LXS components can be varied for optimized delivery as follows: lysophosphatidylcholine:monoglyceride:fatty acid (1:4:2, 1:3, 1:2:4). The acyl groups of these components can also be varied in saturation:unsaturation to affect a solid, semi-solid, or liquid LXS composition at room temperature. The final molar ratios of LXS:drug can range from 1:0.5 to 1:0.9. Briefly, the LXS and solid drug are heated, up to 100-120° C. if needed, to dissolve the drug, resulting in a clear viscous solution. If the drug does not immediately dissolve, a second method of incorporating the drug is evaluated. Generally, the LXS components or LXS matrix are dissolved in an organic solvent (for example, chloroform:methanol, 20:1, v/v), and the neat drug added with low heat until dissolved. The solvents are then removed under vacuum and heat to result in a clear viscous solution. The stability of the LXS/drug eutectic matrix can be evaluated as follows: upon standing overnight at room temperature the LXS/drug matrix should remain clear indicating a stable formulation. If drug crystals appear, then other LXS compositions, LXS containing bound water, and/or other methods of incorporating the drug are evaluated at a lower LXS:drug molar drug. (It should be recognized that LXS containing greater than 1 mole of water forms a lamellar organization and LXS containing 6-8 moles of bound water forms an inverse hexagonal structure.) Once a stable LXS/drug matrix is obtained, then the LXS/drug matrix is sonicated in sodium bicarbonate solution and then subjected to size exclusion chromatography. The LXS/drug matrix (approximately 70 nm) will elute first from the column and any free drug, if any, will elute later. LXS/drug formulations that are stable generally have good/excellent bioavailability in animals and humans.

Further, PPMP can be formulated for co-delivery with 4-HPR in NCI Diluent-12. Formulation of PPMP for intravenous delivery.

We have found that much higher plasma and tissue levels of 4-HPR can be obtained by intravenous delivery of 4-HPR compared to oral delivery. Said intravenous formulations of 4-HPR obtain significantly higher 4-HPR plasma (50-150 μM) and tissue levels in rodent and canine animal models than the current oral formulation while retaining minimal systemic toxicity as described herein.

PPMP can be formulated for intravenous delivery in Diluent-12. Diluent-12 (50% Cremophor EL/50% ethanol) is used clinically as a vehicle for Taxol and cyclosporine A. It has the disadvantage of being castor bean oil-based, necessitating pre-medication to reduce allergic reactions. However, as demonstrated, this method can be used if needed. PPMP, however, can be emulsified, using Lipoid E 80, or other similar vehicle.

Said intravenous and oral formulations will allow pre-clinical modeling of the bioavailability and anti-tumor activity of each route using small animal pharmacokinetic and tumor xenograft models. Respective formulations may also each have separate advantages in the hospital vs. home treatment setting. Further, intravenous and/or oral formulations of PPMP developed as set forth herein will achieve plasma and tissue levels in vivo that effectively inhibit catabolism of 4-HPR-induced ceramide in vitro, will prove of tolerable toxicity, and will enhance 4-HPR anti-tumor activity in vivo.

In the following examples, the DIMSCAN assay is a cell survival assay, not merely an apoptosis assay, and therefore reflects cancer cell killing due to both apoptosis and necrosis. DIMSCAN correlates directly with more traditional clonogenic assays. Moreover, DIMSCAN drug resistance profiles of cell lines correlate with prior patient therapy. DIMSCAN has successfully predicted clinical activity in high-risk neuroblastoma patients for the following new agents: 13-cis-retinoic acid, BSO+L-PAM, and fenretinide. Cytotoxicity assays are performed in 96 well microplates using a semi automated Digital Image Microscopy (DIMSCAN) system that has a dynamic range of greater than 4-5 logs of cell kill. Briefly, following incubation with study drugs, fluorescein diacetate [10 μg/ml (a vital stain)] is added to the microplate and incubated for twenty minutes. Eosin-Y (800 μg/ml) is then added to quench background fluorescence in the medium and non-viable cells. The plates are then read on an inverted microscope with the relative fluorescence of each well determined by the video imaging system software designed for the DIMSCAN system. We have done comparison studies and have shown that the relative fluorescent values obtained by DIMSCAN correlate to cell density (standard counts by trypan blue exclusion) and clonogenicity assays. The present invention is explained in greater detail in the following non-limiting examples.

### EXAMPLE 1

### 4-HPR is Cytotoxic to Solid Tumor and Acute Lymphoblastic Leukemia (ALL) Cell Lines In Vitro

As described above, 4-HPR may cause cytotoxicity in cell lines of many tumor cell types in vitro. During investigations to determine the potential of 4-HPR to treat alkylator- and retinoic acid-resistant neuroblastoma cell lines, we have found that 4-HPR caused less than 1 to 4 logs of cell killing in cell lines of pediatric neuroblastoma and PNET/Ewing's sarcoma, and in multiple adult solid tumors, including lung, breast, colon, melanoma, and pancreas in vitro. In neuroblastoma cell lines, 4-HPR cytotoxicity was p53-, and partially caspase-independent, and induced cell killing by a mixed apoptosis/necrosis. We also found that 4-HPR was cytotoxic to multiple pediatric ALL cell lines.

### EXAMPLE 2

### 4-HPR increased ceramide in solid tumor and ALL leukemia cell lines

4-HPR has been reported to increase Reactive Oxygen Species (ROS) in certain, but not all, solid tumor cell lines and leukemia cell lines in vitro as a mechanism of cytotoxicity. We have found that 4-HPR increased ROS in two neuroblastoma cell lines. However, antioxidants minimally reduced 4-HPR cytotoxicity in neuroblastoma cell lines, particularly at higher 4-HPR dose levels. These results suggest that ROS is only partially responsible for 4-HPR cytotoxicity in neuroblastoma cell lines, particularly at higher dose levels. We, therefore, have explored alternative mechanisms of 4-HPR cytotoxicity. We determined that 4-HPR significantly increased ceramide, up to 13-fold, in a dose- and time-dependent manner in cell lines of neuroblastoma, and in PNET/Ewing's sarcoma, breast, and lung cancer cell lines in vitro. Further, we have found that the increase in ceramide began early (less than 2 hrs post exposure), was progressive with time, and considerably preceded morphological evidence of cell death. We demonstrated that 4-HPR also greatly increased ceramide in multiple ALL cell lines. These data demonstrated that the increase of ceramide stimulated by 4-HPR treatment was not caused as a result of late cell death processes, and raised the possibility that the increase of ceramide may have been contributory 4-HPR cytotoxicity.

### EXAMPLE 3

### 4-HPR Increased Ceramide by Stimulation of de novo Synthesis

We have determined in solid tumor and ALL cell lines in vitro, that the ceramide increased by 4-HPR treatment was derived from de novo synthesis. Radiolabeling experiments demonstrated that membrane sphingomyelin was not decreased by 4-HPR treatment. In contrast, inhibitors of de novo ceramide synthesis, such as L-cycloserine and fumonisin B, prevented the increase of ceramide caused by 4-HPR treatment. Further, 4-HPR stimulated the activities of both serine palmitoyltransferase, the rate-limiting enzyme of de novo ceramide synthesis, and of ceramide synthase (as shown in FIG. 1), by direct assay of enzymatic activity.

### EXAMPLE 4

### 4-HPR was Minimally Cytotoxic in Normal Cells, and Non-Transformed Cell Lines

Having established the potential of high-dose 4-HPR to treat resistant neuroblastoma, in order to investigate the therapeutic index of 4-HPR, we examined the cytotoxicity of 4-HPR in normal and non-malignant cell lines in vitro. We determined that doses of 4-HPR that caused cytotoxicity to multiple types of cancer cell lines in vitro, were minimally toxic to normal fibroblasts and normal bone marrow myeloid progenitors, and to normal peripheral blood mononuclear cells, and an EBV-immortalized, but non-malignant, lymphoblastoid cell line. Accordingly, 4-HPR cytotoxicity is most likely a malignancy-specific event, and high-dose 4-HPR may have an acceptable therapeutic index in vivo.

### EXAMPLE 5

### 4-HPR did not Increase Ceramide in Normal Cells and Non-Transformed Cell Lines

Having established that 4-HPR increased ceramide and caused cytotoxicity in cell lines of a variety of tumor cell types, we examined if 4-HPR increased ceramide in normal cells and non-transformed cell lines. We determined that 4-HPR only minimally increased ceramide in normal fibroblasts, and in normal peripheral blood mononuclear cells and an EBV-immortalized, but not transformed, lymphoid cell line. Accordingly, the ability of 4-HPR to increase ceramide by de novo synthesis is most likely a malignancy (transformed phenotype)-specific event. Further, high-dose 4-HPR will have a favorable therapeutic index in vivo. Moreover, second agents that inhibit ceramide catabolism will also have a favorable therapeutic index in combination with 4-HPR, as normal tissues will not increase ceramide in response to 4-HPR.

### EXAMPLE 6

### 4-HPR Cytotoxicity Correlates with Ceramide Level

As ROS did not account for all of the cytotoxicity induced by high-dose 4-HPR, we considered the role of de novo ceramide in 4-HPR cytotoxicity. Referring to FIG. 2, the cytotoxicity of high-dose 4-HPR correlated with the amount of increase of ceramide. Normal human fibroblasts and neuroblastoma cell lines were exposed to 4-HPR. Ceramide levels were assayed at +24 hours. Cytotoxicity was assayed by DIMSCAN at +96 to 120 hours. As shown in FIG. 2, 4-HPR-sensitive cell lines had higher ceramide levels a +24 hours.

### EXAMPLE 7

### 4-HPR-induced ceramide mediates cytotoxicity

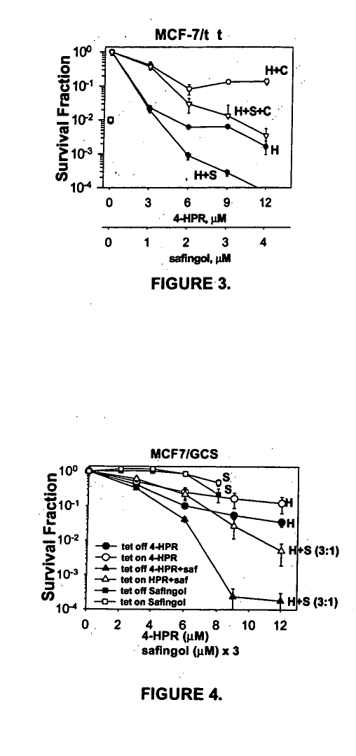

As ROS did not account for all of 4-HPR's cytotoxicity at higher doses, we explored alternative mechanisms of cytotoxicity. We observed that: 1) large, novel increases of ceramide (up to thirteen-fold) occurred by de novo synthesis in a time- and dose-dependent manner, 2) ceramide increase considerably preceded morphological evidence of cell death, 3) ceramide increase was minimal in normal human cells and non-malignant cell lines to which 4-HPR is non-toxic, and 4) 4-HPR cytotoxicity correlated with the magnitude of ceramide increase (as shown in FIG. 2). Accordingly, ceramide increase most likely contributed to 4-HPR cytotoxicity. To further investigate the role of ceramide in 4-HPR cytotoxicity, we tested the effects of inhibitors of de novo ceramide synthesis on 4-HPR cytotoxicity alone, and in combination with safingol (L-threo-dihydrosphingosine), a ceramide-modulating agent that significantly synergizes 4-HPR cytotoxicity in many cell lines. Referring to FIG. 3, while L-cycloserine and fumonisin B proved toxic of themselves to neuroblastoma cell lines, L-cycloserine prevented ceramide increase and significantly decreased the cytotoxicity of 4-HPR, and of 4-HPR+safingol, in MCF-7/tet, an MCF-7 breast cancer cell line. Further, referring to FIG. 4, overexpression of glucosylceramide synthase (GCS), which shunts de novo ceramide into nontoxic glucosylceramide, using a tetracycline-inducible promoter in MCF-7/GCS cells, reduced ceramide, significantly reduced 4-HPR single-agent cytotoxicity, and virtually abrogated the cytotoxic synergy of 4-HPR+safingol. Accordingly, the ceramide pool increased by 4-HPR is most likely directly cytotoxic to cancer cells, and also that the mechanism of safingol synergy is directly dependent upon ceramide. Further, these results suggest that agents that inhibit the conversion of ceramide to nontoxic glucosylceramide and 1-O-acylceramides, will further increase 4-HPR-induced ceramide and cytotoxicity.

Referring to FIG. 3, L-cycloserine is an inhibitor of serine palmitoyltransferase (SPT). MCF-7/tet breast cancer cells exposed to ethanol (controls), 4-HPR(H) or 4-HPR+safingol (3:1 ratio)(H+S), without or with preincubation with L-cycloserine (2 mM)(+C) and assayed by DIMSCAN assay at +96 hrs. 4-HPR (•); 4-HPR/L-cycloserine (O); 4-HPR/safingol (3:1)(▾); 4-HPR/safingol/L-cycloserine (∇); L-cycloserine (2 mM) (□). L-cycloserine reduced the cytotoxicity of 4-HPR and 4-HPR/safingol when normalized to L-cycloserine-treated controls. Accordingly, de novo ceramide is contributory to single agent 4-HPR cytotoxicity, and to safingol cytotoxic synergy.

Referring to FIG. 4, GCS was transfected on a tet-inducible expression vector and induced with 3 μM doxycycline. Ceramide generation by 4-HPR has been shown to be dose-dependent in neuroblastoma cells. Overexpression of GCS shunts ceramide (toxic) to glucosylceramide (nontoxic) and confers doxorubicin resistance. Overexpression of GCS has minimal impact on safingol as a single agent, decreases cytotoxicity of 4-HPR as a single agent (consistent with mixed cytotoxocity due to ROS and ceramide), but almost entirely eliminates the cytotoxic synergy of 4-HPR+safingol (3:1 molar ratio), meaning that 4-HPR+safinogl cytotoxic synergy is ceramide-dependent. Statistical analysis is by two-sided Student's t-test: P<0.0001 at 6 μM H+S; P<0.0001 at 9 μM H+S; P=0.0002 at 12 μM H+S.

### EXAMPLE 8

### Intravenous 4-HPR Obtains High Drug Levels

In order to maximize the potential of 4-HPR to increase ceramide in tumors clinically, it is likely that high, sustained levels of 4-HPR will be needed. Utilizing intravenous formulations, we have directly tested continuous venous infusion (c.i.v.) in rats. Results shown in Table 1 demonstrate that c.i.v. delivery of 4-HPR resulted in high, sustained levels of 4-HPR in plasma and tissues. We have also determined, that safingol (L-threo-dihydrosphinganine), a putative inhibitor of PKC-ζ and sphingosine kinase, can significantly increase the anti-tumor activity of 4-HPR in human neuroblastoma murine xenograft models. Accordingly, the anti-tumor activity of 4-HPR will be increased by other agents which modulate the metabolism of ceramide, such as inhibitors of glucosylceramide synthase and 1-O-acylceramide synthase.

### EXAMPLE 9

Glucosylceramide and 1-O-acylceramide Synthesis Inhibitors

One cellular mechanism to reduce ceramide cytotoxicity is to shunt it into nontoxic forms, such as glucosylceramides and 1-O-acylceramides (as shown in FIG. 1). Increased levels of glucosylceramides are associated with doxorubicin-resistance in MCF-7 breast cancer cells in vitro, and pharmacologic inhibitors of glucosylceramide synthase (GCS), or GCS-antisense expression, restore doxorubicin-induced ceramide levels, and reverse drug resistance. D,L-threo-(1-phenyl-2-hexadecanoylamino-3-morpholino-1-propanol) (PPMP) is reported to inhibit both GCS and 1-O-acylceramide synthase. In contrast, D,L-erythro-PPMP inhibits only 1-O-acylceramide synthase activity, and increased cellular ceramide without decreasing glucosylceramide levels. PPMP is a more active congener of related compound PDMP, which is well tolerated in rodents to doses of 120 mg/kg/day x 10 days, and can achieve cures of Ehrlich ascites tumor cells xenografts in vivo. Further, we have reported that D,L-PPMP can increase the cytotoxicity of 4-HPR and 4-HPR+safingol in solid tumor cell lines, and of 4-HPR in ALL cell lines, in vitro.

### EXAMPLE 10

Glucosylceramide Synthase (GCS) and 1-O-acylceramide Synthase (1-O-ACS) are Widely Expressed in Neuroblastoma and ALL and AML Leukemia Cell Lines

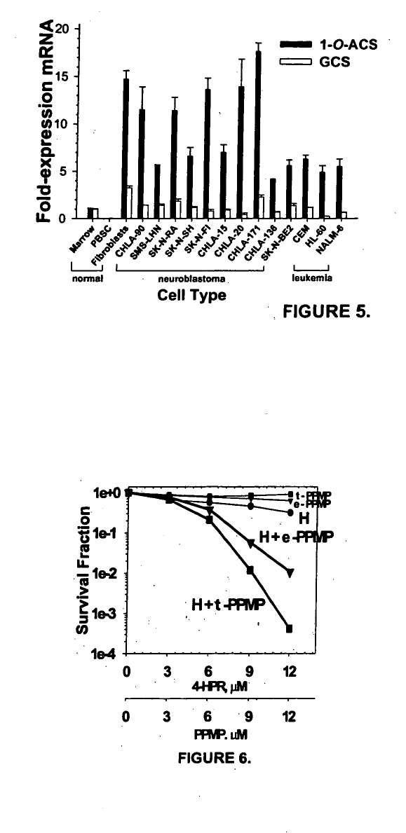

To validate GCS and 1-O-ACS as targets for inhibition, we determined the level of mRNA expression of these enzymes by semi-quantitative PCR assay in a cell line panel of neuroblastoma, leukemias, and normal cells. Referring to FIG. 5, the results demonstrate that both GCS and 1-O-ACS have mRNA expression in many cell lines of these cancer types. These results support other reports that these enzymes are widely expressed in normal tissues. Because these results demonstrate that these enzymes are highly expressed in both a solid tumor (neuroblastoma) and in both acute lymphoblastic (ALL) and acute myelogenous (AML) leukemia cell lines, it is most likely that they will have wide expression in other cancer types, as well.

FIG. 5 shows mRNA expression of GCS and 1-O-ACS. Taqman PCR assay was used to quantitate mRNA levels of GCS and 1-O-ACS in normal cells (marrow, peripheral blood mononuclear cells (PBSC) and normal fibroblasts), neuroblastoma cell lines and acute leukemia cell lines. Results are normalized to that of marrow cells. The results show that these enzymes are expressed in these cancer types and validate GCS and 1-O-ACS as therapeutic targets.

### EXAMPLE 111

### PPMP increased ceramide and reversed 4-HPR-resistance in a neuroblastoma cell line

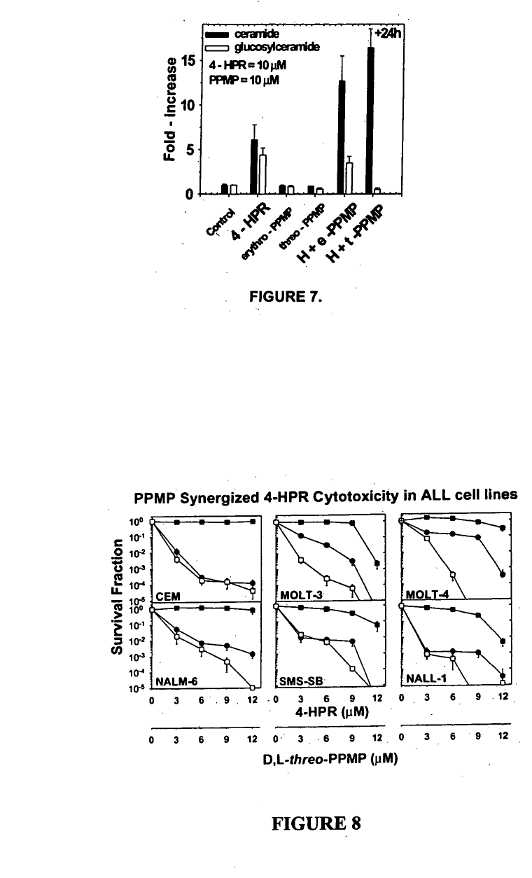

4-HPR only modestly increased ceramide in 4-HPR-resistant SK-N-RA neuroblastoma in vitro. D,L-threo-PPMP inhibits both glucosylceramide synthase and 1-O-acylceramide synthase, whereas D,L-erythro-PPMP inhibits only 1-O-acylceramide synthase. To study the mechanism of 4-HPR resistance in SK-N-RA cells, we exposed SK-N-RA cells to either 4-HPR-alone, 4-HPR+D,L-erythro-PPMP, or 4-HPR+D,L-threo-PPMP. We hypothesized that if 4-HPR resistance was due to active shunting of ceramide into nontoxic glucosylceramide and acylceramides, then both D,L-erythro-PPMP and D,L-threo-PPMP would increase ceramide and 4-HPR cytotoxicity, but that D,L-threo-PPMP, by virtue of inhibiting both pathways, would be particularly synergistic. We observed that D,L-erythro-PPMP did increase 4-HPR-induced ceramide and cytotoxicity, but that D,L-threo-PPMP more strongly synergized 4-HPR-induced ceramide and 4-HPR cytotoxicity (as shown in FIGS. 6 and 7). Thus, inhibitors of ceramide catabolism, such as PPMP, can increase 4-HPR-induced ceramide and synergize 4-HPR cytotoxicity in cancer cell lines with active glucosylceramide and acylceramide synthase pathways.

FIG. 6 shows PPMP synergized 4-HPR cytotoxicity in a resistant neuroblastoma cell line. Survival fraction was measured using a digital imaging fluorescence-based microscopy assay (DIMSCAN) with approximately 5 log sensitivity. Assayed at +96 h. All three drugs were minimally- or nontoxic separately in SK-N-RA neuroblastoma cells. Both drugs reversed resistance and synergized 4-HPR cytotoxicity (C.I.<1), but D,L-threo-PPMP did so more potently. D,L-erythro-PPMP (e-PPMP) is an inhibitor of 1-O-acylceramide synthase. D,L-threo-PPMP (t-PPMP) is an inhibitor of both glucosylceramide synthase and 1-O-acylceramide synthase. Measure of cytotoxic synergy was by Combination Index (C.I.) methodology: synergy, C.I.<1; additive, C.I.=1; antagonism, (C.I.)>1.

FIG. 7 shows that PPMP further increased 4-HPR-induced ceramide in a multi-drug-resistant neuroblastoma cell line. Ceramide and glucosylceramide levels were measured by labeling with [³H]-palmitic acid and thin-layer chromatography. Assays were performed at +24 h. 4-HPR (10 μM) modestly increased ceramide in SK-N-RA neuroblastoma cells. D,L-erythro-PPMP (e-PPMP), an inhibitor of 1-O-acylceramide synthase, did not affect 4-HPR-induced glucosylceramide levels (P equal to 0.27) but further increased 4-HPR-induced ceramide (P equal to 0.03). D,L-threo-PPMP (t-PPMP), an inhibitor of both glucosylceramide synthase and 1-O-acylceramide synthase, prevented glucosylceramide formation (P equal to 0.01) and even more strongly increased 4-HPR-induced ceramide levels (P equal to 0.002). Statistical analysis is by student's t-test.

### EXAMPLE 12

### PPMP Synergized 4-HPR Cytotoxicity in ALL Cell Lines

We have found that 4-HPR caused multi-log cytotoxicity and significantly increased ceramide by de novo synthesis in a time- and dose-dependent manner in all six tested ALL cell lines in vitro. Given our results with PPMP in solid tumor cell lines, we hypothesized that inhibitors of ceramide catabolism, such as D,L-threo-PPMP, would also synergize 4-HPR cytotoxicity in ALL cell lines. We determined that D,L-threo-PPMP decreased glucosylceramide formation, increased ceramide in the cell lines examined, and synergized 4-HPR cytotoxicity in four of six pediatric ALL cell lines.

FIG. 8 shows that D,L-threo-PPMP synergistically increased 4-HPR cytotoxicity in ALL cell lines. D,L-PPMP synergized 4-HPR cytotoxicity in four of six ALL cell lines. Assayed by DIMSCAN at +96 hours. •=4-HPR; ▪=PPMP; □=4-HPR+PPMP; (1:1 molar ratio). Measure of cytotoxic synergy, Combination Index (C.I.): synergy C.I.<1; additive, C.I.=1; antagonism, C.I.>1; CEM, C.I.=1; MOLT-3, C.I.<1; MOLT-4, C.I.<1; NALM-6, C.I.<1; SMS-SB, 3 μM (C.I.>1), 6 μM (C.I.>1), 9 μM (C.I.=1); NALL-1, 3 μM (C.I.>1), 6 μM (C.I.=1), 9 μM (C.I.<1).

### EXAMPLE 13

### D-threo-PPMP is the most Active PPMP Stereoisomer

Further we have found that D-threo-PPMP is the most active PPMP stereoisomer. Our initial studies were conducted using racemic D,L-PPMP, as it is reported to be 10-20 times as active as PDMP. As observed in FIG. 6 and FIG. 7, D,L-threo-PPMP was more active than D,L-erythro-PPMP. However, the individual enantiomers (i.e., L-threo- and D-threo) of the parent compound, PDMP, have different inhibitory activities when employed separately. Specifically, D-threo-PDMP inhibited GCS and 1-O-ACS activity, decreasing glucosylceramide and increasing ceramide levels, whereas, L-threo-PDMP paradoxically elevated glycosphingolipid levels. Descriptions of these investigations have not been reported for the enantiomers of threo-PPMP. Therefore, we determined the effects of D-threo-PPMP and L-threo-PPMP, on 4-HPR-induced glucosylceramide, ceramide, and cytotoxicity, in a neuroblastoma, a leukemia, and a prostate cancer cell line.

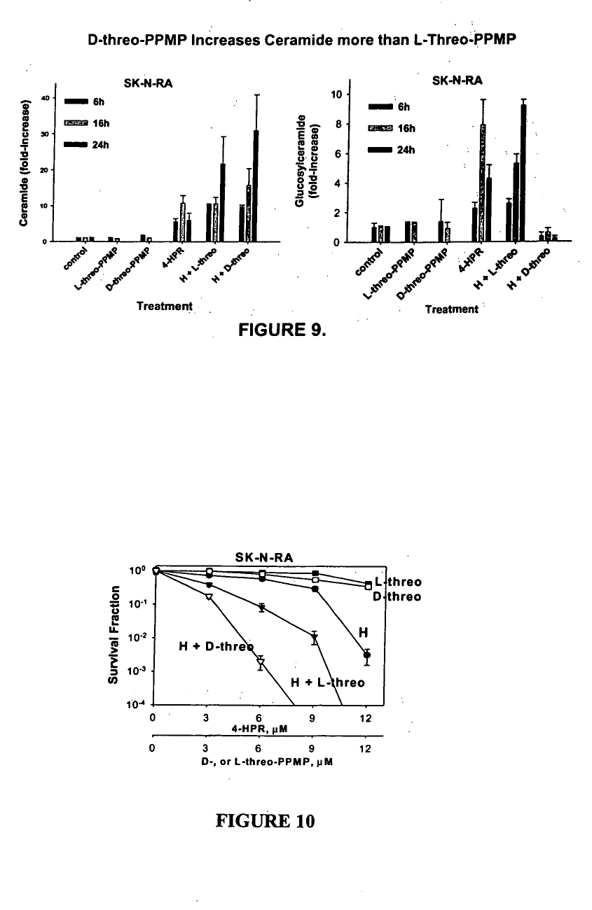

Referring to FIG. 9, preliminary results in SK-N-RA neuroblastoma cells show that D-threo-PPMP inhibits glucosylceramide synthesis, and increases ceramide to a greater degree, than does L-threo-PPMP. SK-N-RA neuroblastoma cells were exposed to drugs at 10 μM concentrations for the time indicated. Lipids were assayed by labeling with [³H]-palmitic acid and thin layer chromatography. The left panel shows D-threo-PPMP increased 4-HPR-induced ceramide more than did L-threo-PPMP. The right panel shows that D-threo-PPMP prevented 4-HPR-induced glycosylceramide increase whereas L-threo-PPMP did not. Together, this shows that both enantiomers may inhibit 1-O-ACS, but unexpectedly, that only D-threo-PPMP inhibits GCS activity. Thus, unexpectedly, D-threo-PPMP, as a single agent, proved to be better at increasing 4-HPR-induced ceramide levels than all other PPMP stereoisomers. Further, as PPMP is 10-20 times more active than PDMP, D-threo-PPMP is unexpectedly the most preferred stereoisomer of all PPMP and PDMP compounds.

Referring to FIG. 10, D-threo-PPMP was more active in synergizing 4-HPR cytotoxicity than is L-threo-PPMP. SK-N-RA neuroblastoma cells were exposed to drug as indicated for +96 hours and results assayed by DIMSCAN. H=4-HPR; L-threo=L-threo-PPMP; D-threo=D-threo-PPMP. Results show that D-threo-PPMP synergizes 4-HPR cytotoxicity more potently than does L-threo-PPMP. These results correlate with the results of ceramide data shown in FIG. 9.

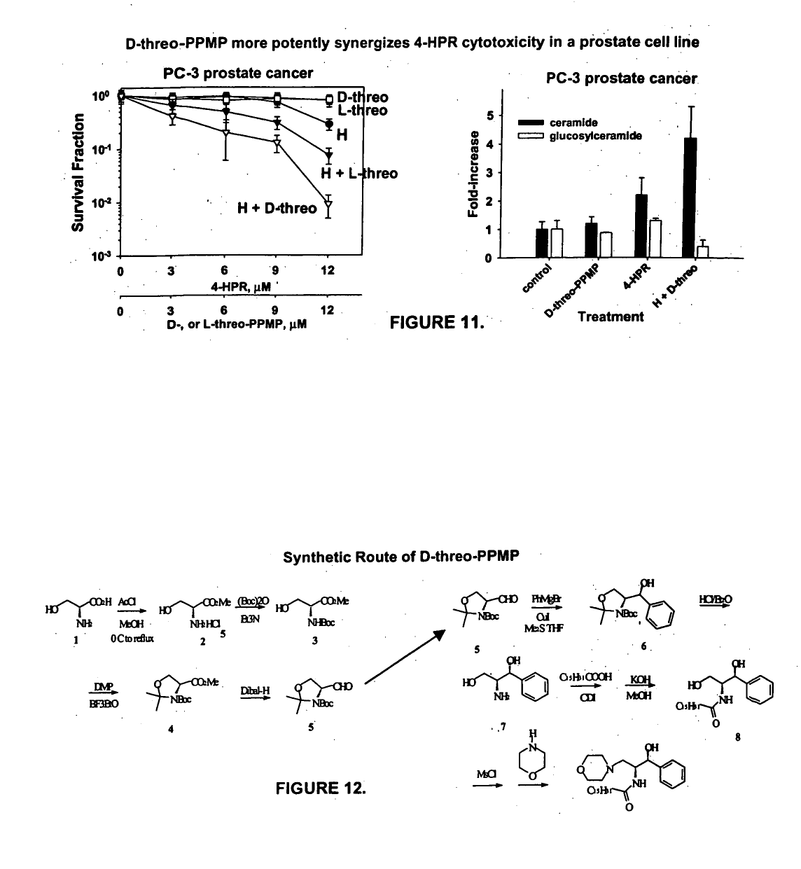

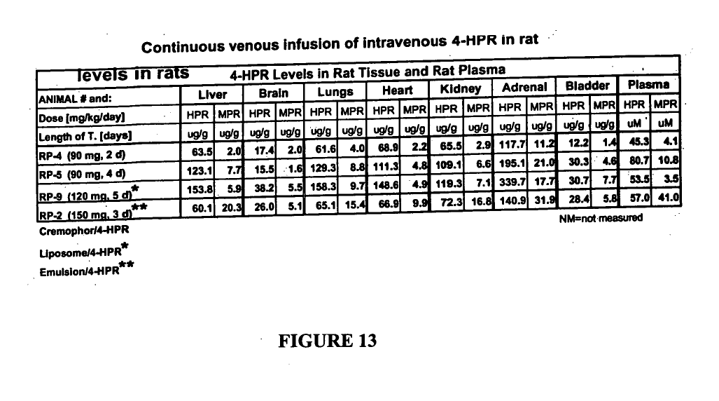

Similar results were observed in BM185 mouse ALL leukemia cells. Referring to FIG. 11, results also show that D-threo-PPMP increased ceramide and more effectively increased 4-HPR cytotoxicity in PC-3 prostate cancer cells. Together, these results show that it is D-threo-PPMP, rather than L-threo-PPMP, that is the superior ceramide degradation inhibitor.

FIG. 11 shows that D-threo-PPMP more potently synergizes 4-HPR cytotoxicity in a prostate cancer cell line than does L-threo-PPMP. The left panel of FIG. 11 shows PC-3 cells, an androgen-independent, PTEN null, prostate cancer cell line, treated with drug as indicated and cytotoxicity assayed at +96 h by DIMSCAN assay. D-threo-PPMP synergized (C.I.<1) 4-HPR cytotoxicity more strongly than did L-threo-PPMP (P<0.04). The right panel of FIG. 11 shows PC-3 cells treated with drug (10 μM), as indicated. Lipids were assayed by labeling with [³H]-palmitic acid and thin-layer chromatography at +24 h. D-threo-PPMP increased ceramide in 4-HPR-treated cells (P=0.035). Synergy assayed by Combination Index (C.I.): synergy, C.I.<1; additive, C.I.=1; antagonism, C.I.>1. Statistical analysis is by student's t-test.

Although the invention has been described with respect to specific embodiments and examples, it will be readily appreciated by those skilled in the art that modifications and adaptations of the invention are possible without deviation from the spirit and scope of the invention. Accordingly, the scope of the present invention is limited only by the following claims.

1. A method of treating a hyperproliferative disorder comprising administering:
a ceramide-generating anticancer agent or treatment; and
a ceramide degradation inhibitor or a pharmaceutically acceptable salt or ester thereof;
wherein the hyperproliferative disorder is a tumor; and
wherein the ceramide-generating anticancer agent or treatment is administered in an amount effective to produce necrosis, apoptosis or both in the tumor, and the ceramide degradation inhibitor is administered in an amount effective to increase the necrosis, apoptosis or both in the tumor over that expected to be produced by the sum of that produced by the ceramide-generating anticancer agent or treatment and the ceramide degradation inhibitor when administered separately.

2. The method of claim 1 wherein the ceramide degradation inhibitor comprises a compound effective in inhibiting glucosylceramide synthase and 1-O-acylceramide synthase.

3. The method of claim 2 wherein the ceramide degradation inhibitor comprises D-threo-PPMP.

4. The method of claim 3 wherein the ceramide-generating anticancer agent or treatment comprises a ceramide generating retinoid comprising a retinoic acid derivative or a pharmaceutically acceptable salt or ester thereof.

5. The method of claim 4 wherein the ceramide generating retinoid comprises fenretinide or a pharmaceutically acceptable salt or ester thereof.

6. The method of claim 5 wherein the ceramide generating retinoid and the ceramide degradation inhibitor are administered intravenously, orally, or topically.

7. A method of treating a hyperproliferative disorder comprising administering:
a ceramide-generating anticancer agent or treatment; and
a ceramide degradation inhibitor or a pharmaceutically acceptable salt thereof effective in inhibiting glucosylceramide synthase and 1-O-acylceramide synthase;
wherein the hyperproliferative disorder is a tumor; and wherein the ceramide generating retinoid is administered in an amount effective to produce necrosis, apoptosis or both in the tumor, and the ceramide degradation inhibitor is administered in an amount effective to increase the necrosis, apoptosis or both in the tumor over that expected to be produced by the sum of that produced by the ceramide-generating anticancer agent or treatment and the ceramide degradation inhibitor when administered separately.

8. The method of claim 7 wherein the ceramide degradation inhibitor comprises D-threo-PPMP.

9. The method of claim 8 wherein the ceramide-generating anticancer agent or treatment comprises a ceramide generating retinoid comprising a retinoic acid derivative or a pharmaceutically acceptable salt or ester thereof.

10. The method of claim 9 wherein the ceramide generating retinoid comprises fenretinide or a pharmaceutically acceptable salt or ester thereof.

11. The method of claim 10 wherein the ceramide generating retinoid and the ceramide degradation inhibitor are administered intravenously, orally, or topically.

12. A method of treating a hyperproliferative disorder comprising administering:
a ceramide generating retinoid comprising fenretinide or a pharmaceutically acceptable salt or ester thereof; and
a ceramide degradation inhibitor comprising D-threo-PPMP or a pharmaceutically acceptable salt or ester thereof;
wherein the hyperproliferative disorder is a tumor; and
wherein the ceramide generating retinoid is administered in an amount effective to produce necrosis, apoptosis or both in the tumor, and the ceramide degradation inhibitor is administered in an amount effective to increase the necrosis, apoptosis or both in the tumor over that expected to be produced by the sum of that produced by the ceramide generating retinoid and the ceramide degradation inhibitor when administered separately.

13. The method of claim 12 wherein the ceramide degradation inhibitor consisting essentially of D-threo-PPMP or a pharmaceutically acceptable salt or ester thereof.

14. The method of claim 13 wherein the ceramide generating retinoid and the ceramide degradation inhibitor are administered intravenously, orally or topically.

15. A formulation for treating a hyperproliferative disorder comprising:
a ceramide-generating anticancer agent or treatment; and
a ceramide degradation inhibitor or a pharmaceutically acceptable salt or ester thereof;
wherein the hyperproliferative disorder is a tumor; and
wherein the ceramide generating anticancer agent or treatment is administered in an amount effective to produce necrosis, apoptosis or both in the tumor, and the ceramide degradation inhibitor is administered in an amount effective to increase the necrosis, apoptosis or both in the tumor over that expected to be produced by the sum of that produced by the ceramide-generating anticancer agent or treatment and the ceramide degradation inhibitor when administered separately.

16. The formulation of claim 15 wherein the ceramide degradation inhibitor comprises a compound effective in inhibiting glucosylceramide synthase and 1-O-acylceramide synthase.

17. The formulation of claim 16 wherein the ceramide degradation inhibitor comprises D-threo-PPMP or a pharmaceutically acceptable salt or ester thereof.

18. The formulation of claim 17 wherein the ceramide-generating anticancer agent or treatment comprises a ceramide generating retinoid comprising a retinoic acid derivative or a pharmaceutically acceptable salt or ester thereof.

19. The formulation of claim 18 wherein the ceramide generating retinoid comprises fenretinide or a pharmaceutically acceptable salt or ester thereof.

20. The formulation of claim 19 wherein the ceramide generating retinoid and the ceramide degradation inhibitor are administered intravenously, orally, or topically.

21. A formulation for treating a hyperproliferative disorder comprising:
a ceramide generating retinoid comprising fenretinide or a pharmaceutically acceptable salt or ester thereof; and
a ceramide degradation inhibitor comprising D-threo-PPMP or a pharmaceutically acceptable salt or ester thereof;
wherein the hyperproliferative disorder is a tumor; and wherein the ceramide generating retinoid is administered in an amount effective to produce necrosis, apoptosis or both in the tumor and the ceramide degradation inhibitor is administered in an amount effective to increase the necrosis, apoptosis or both in the tumor over that expected to be produced by the sum of that produced by the ceramide generating retinoid and the ceramide degradation inhibitor when administered separately.

22. The formulation of claim 21 wherein the ceramide degradation inhibitor consisting essentially of D-threo-PPMP or a pharmaceutically acceptable salt or ester thereof.

23. The formulation of claim 21 wherein said formulation is administered intravenously, orally, or topically.

24. A method of treating a hyperproliferative disorder comprising administering:
a ceramide-generating anticancer agent or treatment; and
a ceramide degradation inhibitor wherein said ceramide degradation inhibitor comprises D-threo-PPMP or a pharmaceutically acceptable salt or ester thereof; wherein the hyperproliferative disorder is a tumor; and
wherein the ceramide-generating anticancer agent or treatment is administered in an amount effective to produce necrosis, apoptosis or both in the tumor, and D-threo-PPMP is administered in an amount effective to increase the necrosis, apoptosis or both in the tumor over that expected to be produced by the sum of that produced by the ceramide-generating anticancer agent or treatment and D-threo-PPMP when administered separately.

25. The method of claim 24 wherein the ceramide degradation inhibitor consisting essentially of D-threo-PPMP or a pharmaceutically acceptable salt or ester thereof.

26. A formulation for treating a hyperproliferative disorder comprising:
a ceramide-generating anticancer agent or treatment; and
a ceramide degradation inhibitor wherein said ceramide degradation inhibitor comprises D-threo-PPMP or a pharmaceutically acceptable salt or ester thereof;
wherein the hyperproliferative disorder is a tumor; and wherein the ceramide-generating anticancer agent or treatment is administered in an amount effective to produce necrosis, apoptosis or both in the tumor, and D-threo-PPMP is administered in an amount effective to increase the necrosis, apoptosis or both in the tumor over that expected to be produced by the sum of that produced by the ceramide-generating anticancer agent or treatment and D-threo-PPMP when administered separately.

27. The formulation of claim 26 wherein the ceramide degradation inhibitor consisting essentially of D-threo-PPMP or a pharmaceutically acceptable salt or ester thereof.

28. A method of treating a hyperproliferative disorder comprising administering:
a ceramide-generating anticancer agent or treatment; and
a ceramide degradation inhibitor wherein said ceramide degradation inhibitor comprises a single isomer that is effective in inhibiting both glucosylceramide synthase and 1-O-acylceramide synthase;
wherein the hyperproliferative disorder is a tumor; and wherein the ceramide-generating anticancer agent or treatment is administered in an amount effective to produce necrosis, apoptosis or both in the tumor, and the ceramide degradation inhibitor is administered in an amount effective to increase the necrosis, apoptosis or both in the tumor over that expected to be produced by the sum of that produced by the ceramide-generating anticancer agent or treatment and the ceramide degradation inhibitor when administered separately.

29. The method of claim 28 wherein the isomer comprises D-threo-PPMP or a pharmaceutically acceptable salt or ester thereof.

30. The method of claim 29 wherein the ceramide-generating anticancer agent or treatment comprises a ceramide generating retinoid comprising a retinoic acid derivative or a pharmaceutically acceptable salt or ester thereof.

31. The method of claim 30 wherein the ceramide generating retinoid comprises fenretinide or a pharmaceutically acceptable salt or ester thereof.

32. The method of claim 31 wherein the ceramide generating retinoid and the ceramide degradation inhibitor are administered intravenously, orally, or topically.
